(19) United States
(12) Patent Application Publication
(10) Pub. No.: US 2012/0323134 A1
(43) Pub. Date: Dec. 20, 2012

(54) METHOD AND SYSTEM FOR DETERMINING A LOCATION OF NERVE TISSUE IN THREE-DIMENSIONAL SPACE

(75) Inventor: Philip C. Cory, Bozeman, MT (US)

(73) Assignee: NERVONIX, INC., Bozeman, MT (US)

(21) Appl. No.: 13/501,438

(22) PCT Filed: Feb. 27, 2012
(86) PCT No.: PCT/US12/26775

§ 371 Date: Apr. 11, 2012

Related U.S. Application Data
(60) Provisional application No. 61/447,505, filed on Feb. 28, 2011.

(51) Int. Cl. A61B 5/053

(52) U.S. Cl. 600/547

(57) ABSTRACT

Systems and methods for discriminating and locating nerve tissues within a body involve applying a waveform signal to tissue between two electrodes and measuring the electrical characteristics of the signal transmitted through the tissue. Using impedance measurements, the (x, y) coordinates of a nerve relative to an electrode array on the skin surface, and the z-coordinate of the nerve depth position, may be determined. A controller may implement the process and perform the impedance calculations on the measured data to identify tissue types and locations within the measured area, and to present results in graphical form. Results may be combined with other tissue imaging technologies and with image-guided systems.

### RELATED APPLICATIONS

This application is the national stage of International Application No. PCT/US2012/026775, filed Feb. 27, 2012, which was filed in English and claimed the benefit of priority to U.S. Provisional Patent Application No. 61/447,505 entitled “Nerve-Related Time Constant Extraction from Skin Surface Electrical Parameter Determination,” filed on Feb. 28, 2011. The entire contents of both International Application No. PCT/US2012/026775 and U.S. Provisional Patent Application No. 61/447,505 are hereby incorporated by reference.

### BACKGROUND OF THE INVENTION

1. Field of the Invention

Methods and systems according to the various embodiments relate to non-invasively determining the location of and imaging nerve tissue in three-dimensional space.

2. Description of the Related Art

Non-invasive detection of subcutaneous tissues has concerned medical practitioners for many years. It is known by practitioners that many forms of subcutaneous tissue are responsive to electrical signals. Biologic, electrically responsive membrane systems (BERMS) are lipid bi-layers containing embedded protein molecules, some of which are ion channels. The density of embedded ion channels varies by tissue type, with nerve tissue having the highest concentrations of ion channels per gram of tissue. Nerve abnormalities, e.g., neuromas, have even higher concentrations of ion channels than normal nerve tissue. Other tissues, e.g., muscle, have lower densities than normal nerve tissue.

Nerves appear to demonstrate electrical inductance in an externally applied electrical field. This membrane effect occurs in addition to the widely appreciated membrane resistance and membrane capacitance. Sub-threshold, alternating, electrical fields do not generate action potentials, but cause anomalous impedance (appearing as an inductance), which has been noted and modeled in single axon systems. Mauro, Anomalous Impedance, A Phenomenological Property Of Time-Variant Resistance, An Analytic Review, (The Rockefeller Institute (1961)), proposes a mechanism to explain this anomalous impedance, which is based on the effect of normal membrane currents flowing across the axon membrane in the opposite direction to the applied field. These currents are associated with time variant, ion-specific conductance and behave electrically as inductance. In addition, Sabah and Leibovic, Subthreshold Oscillatory Responses Of The Hodgkin-Huxley Cable Model For The Squid Giant Axon, (Department of Biophysical Sciences, Center for Theoretical Biology, State University of New York at Buffalo, Amherst, N.Y. (1969)), disclose circuit models of membrane electrical inductance, connected in parallel with membrane capacitance and membrane resistance and predict an electrical resonance effect.

Previous noninvasive, electrically based methods for determining, from a skin surface, the tissue depth, composition, configuration, and/or state of function relies on either detecting a change in the function of the biological tissue structure in response to stimulation, or assuming characteristics about electrical field paths in tissue.

U.S. Pat. No. 6,167,304 to Loos discusses the use of induced electrical fields to cause nerve “resonance.” It is unclear specifically what is meant by the term resonance in the Loos disclosure. This resonance occurs at certain frequencies and is associated with physiological findings. However, it is clearly not the same as the electrical phenomenon of resonance, which is a function of inductance and capacitance connected either in series or in parallel, with a resistance resulting in marked impedance changes at a single, unique frequency. The determination of impedance plays no role in the Loos resonance, which occurs at multiple frequencies.

In the technique of EIT, current flow between a pair of electrodes causes simultaneous voltage, amplitude, phase, or waveform variations at other, non-current carrying electrodes arrayed on the body surface or in subcutaneous tissues, as described in U.S. Pat. No. 6,055,452 to Pearlman. Varying the electrode pairs through which current is flowing, followed by combining and analyzing the data, allows construction of specific impedance images that may be related to underlying structures. A key assumption for the performance of EIT is that tissues have unique electrical characterizations, the most important being the specific impedance, tissue resistivity, and tissue dielectric constant. The electrical field itself does not affect these parameters, although changes in organ size, content, conformation, or state of function are reflected in altered conductivity patterns. The technique of EIT analyzes voltage information from the skin surface at points distinct from the current carrying pair of electrodes. The assumption is made that tissue resistivities or dielectric constants are stable in the presence of these electrical fields, allowing the calculation of current flow patterns beneath the skin surface and construction of images from those patterns. In this technique, resolution and identification of subsurface structures remains a problem.

U.S. Pat. No. 5,560,372 to Cory teaches that, under certain conditions, the applied voltage required for maintenance of controlled current flow through skin surface electrodes is reduced when measured on skin over the position of peripheral nerves as compared to skin not overlying significant nerve tissue. This capability has not been addressed with other techniques, e.g., electrical impedance tomography (EIT). The device taught in the '372 patent indicates the lowest impedance site within its field by activating a single light emitting diode (LED) corresponding to the electrode contacting the skin surface at that site.

The recognition that tissue represents a non-homogeneous conductor best modeled as a parallel resistance and capacitance with a series resistance has enabled determination of the bulk conductor electrical properties of tissue. U.S. Pat. No. 7,865,236 to Cory, the entire contents of which are hereby incorporated by reference, teaches that tissue electrical anisotropicities, associated with peripheral nerves, are detectable from the skin surface by the application of electrical fields having specific waveform characteristics, and current and voltage levels below the threshold for generating an action potential. However, while the '236 patent teaches a method for constructing two-dimensional maps of nerve tissue from the calculated bioimpedance, it does not teach a method or system for determining nerve depth. Accordingly, there exists a need to non-invasively locate nerve in the (x, y, z) space of living tissue.

### SUMMARY OF THE INVENTION

The various embodiments provide improved systems, apparatus and methods for accurately locating and discriminating nerve tissue dimensions including depth in three dimensions of living tissue. In an embodiment, a method for discriminating the location of nerve tissue includes monitoring changes in electrical parameters of an applied electrical field induced by localized electrical characteristics of the subject, for example, the presence of the nerve tissue density distribution. In an embodiment, the electrical parameters include impedance.

The various embodiments provide a method for discriminating depth of a nerve beneath a skin surface of a subject that may include: placing a waveform electrode array on the skin of the subject, where the waveform electrode array includes at least one waveform electrode that has an area of approximately 10 mm² or less; placing a return electrode on the skin of the subject at a spacing distance from the at least one waveform electrode such that impedance is minimized, where the spacing distance is greater than a distance at which impedance is maximized; applying at least one electrical signal serially to each of the at least one waveform electrode and the return electrode, where an electrical circuit including tissue of the subject as a component is completed; calculating impedance of the tissue associated with the applied waveform for each of the at least one waveform electrodes; identifying a waveform electrode with a lowest calculated impedance value; discriminating a projected (x, y) position of the nerve beneath the waveform electrode array using the identified waveform electrode having a lowest calculated impedance values; determining a mathematical relationship of impedance (Z) to length (l) for an electrical path to the nerve using known equivalent circuit models; generating a table correlating impedance with nerve depth, in which the length of the electrical path to the nerve is a sum of a first non-hypotenuse leg and a second non-hypotenuse leg of a right triangle, the first non-hypotenuse leg is equal to a distance from the waveform electrode position on the skin and the projected (x, y) position of the nerve, and the second non-hypotenuse leg is equal to the nerve depth; and using the table to determine a nerve depth value for the calculated impedance value of a waveform electrode.

A tissue discrimination system according to a embodiment may include: a waveform generator configured to generate a plurality of different waveforms; a waveform electrode array coupled to the waveform generator, in which the waveform electrode array comprises at least one waveform electrode that is at least 10 mm², and in which the waveform electrode array is configured to apply a waveform to a tissue; at least one return electrode configured to receive the applied waveform from the tissue and to provide the applied waveform to the controller, in which the return electrode is spaced at an inter-electrode distance from the waveform electrode array that minimizes impedance and that is greater than a distance of maximum impedance; and a controller coupled to the waveform generator and the at least one return electrode. The controller of the various embodiments may be configured to perform operations including: causing a waveform to be applied serially to the waveform electrode array and the return electrode, calculating impedance of the tissue associated with the applied waveform for each of the at least one waveform electrodes; identifying a waveform electrode with a lowest calculated impedance value; discriminating a projected (x, y) position of the nerve beneath the waveform electrode array using the identified waveform electrode having a lowest calculated impedance values; determining a mathematical relationship of impedance (Z) to length (l) for an electrical path to the nerve using known equivalent circuit models; generating a table correlating impedance with nerve depth, where the length of the electrical path to the nerve comprises a sum of a first non-hypotenuse leg and a second non-hypotenuse leg of a right triangle, where the first non-hypotenuse leg is equal to a distance from the waveform electrode position on the skin and the projected (x, y) position of the nerve, and where the second non-hypotenuse leg is equal to the nerve depth; and using the table to determine a nerve depth value for the calculated impedance value of a waveform electrode.

The various embodiments may be implemented as a non-transitory computer-readable storage medium having stored thereon processor-executable instructions configured to cause a controller to perform operations including: applying at least one electrical signal serially to each of the at least one waveform electrode and a return electrode, where an electrical circuit including tissue of the subject as a component is completed; calculating impedance of the tissue associated with the applied waveform for each of the at least one waveform electrodes; identifying a waveform electrode with a lowest calculated impedance value; discriminating a projected (x, y) position of the nerve beneath the waveform electrode array using the identified waveform electrode having a lowest calculated impedance values; determining a mathematical relationship of impedance (Z) to length (l) for an electrical path to the nerve using known equivalent circuit models; generating a table correlating impedance with nerve depth, in which the length of the electrical path to the nerve is a sum of a first non-hypotenuse leg and a second non-hypotenuse leg of a right triangle, the first non-hypotenuse leg is equal to a distance from the waveform electrode position on the skin and the projected (x, y) position of the nerve, and the second non-hypotenuse leg is equal to the nerve depth; and using the table to determine a nerve depth value for the calculated impedance value of a waveform electrode

### BRIEF DESCRIPTION OF THE DRAWINGS

The accompanying drawings, which are incorporated in and form a part of the specification, illustrate the various embodiments of the invention and, together with the description, serve to explain the principles of the invention.

FIG. 1 is a representative cross-sectional view of an electrode and underlying nerves with an applied electrical field in an ideal homogeneous medium.

FIG. 2 is a representative cross-sectional view of two electrodes and an underlying nerve with an applied electrical field in an ideal homogeneous medium.

FIG. 3 is a representative cross-sectional view of two electrodes and an underlying nerve with an applied electrical field in a non-homogeneous medium.

FIG. 4 is a representative cross-sectional view showing a model for the electrical path through tissue including a nerve.

FIG. 5 is a representative cross-sectional view showing a model of axons of a nerve electrically interacting with an embodiment tissue discrimination system.

FIG. 6 is a graph showing observed impedance data of signals through electrodes as a function of electrode contact surface area.

FIG. 7A is a graph showing resistance data of signals through electrodes as a function of current density and signal frequency.

FIG. 7B is a graph showing capacitance data of signals through electrodes as a function of current density and signal frequency.

FIG. 8 is a graph showing the relationship between tissue impedance Z and electrode separation distance D for a fixed frequency of an applied electrical field.

FIG. 9 is a component block diagram of an embodiment system for discriminating tissues.

FIG. 10 is a component block diagram for modeling tissue as an R-C circuit element according to an embodiment.

FIG. 11 is a graph showing impedance measurements versus frequency for tissue containing high density of nerve tissue.

FIG. 12 is a graph showing impedance measurements versus frequency for tissue containing low density of nerve tissue.

FIG. 13 is a graph showing impedance calculations at a fixed frequency over a plurality of RC circuits having capacitances of 1×10⁻⁸ farads and differing resistances.

FIG. 14 is a process flow diagram of an embodiment method for detecting the effect of electrical resonance on impedance values.

FIG. 15 is a graph showing two voltage decay curves from delivery of a controlled current, square waveform pulse to a tissue via needle electrodes of different diameters.

FIG. 16 is a process flow diagram of an embodiment method of determining a projected (x, y) position for the nerve.

FIG. 17 is a representative cross-sectional view of a two-dimensional waveform electrode array on skin of a subject and an underlying nerve position.

FIG. 18 is a representative cross-sectional view and corresponding graph of sensed impedance value for a linear series of electrodes on skin overlying a nerve.

FIG. 19 is a representative cross-sectional view of a right triangle between a waveform electrode on skin, and a projected (x, y) position of a nerve, and the depth of the nerve in the z plane.

FIG. 20 is a circuit diagram of an equivalent RLC circuit model suitable for modeling electrical responses of tissues for use in the various embodiments.

### DETAILED DESCRIPTION OF THE INVENTION

The various embodiments will be described in detail with reference to the accompanying drawings. Wherever possible, the same reference numbers will be used throughout the drawings to refer to the same or like parts. References made to particular examples and implementations are for illustrative purposes and are not intended to limit the scope of the invention or the claims.

The word “exemplary” is used herein to mean “serving as an example, instance, or illustration.” Any implementation described herein as “exemplary” is not necessarily to be construed as preferred or advantageous over other implementations.

In the various embodiment methods, a waveform may be generated and provided to tissue of a subject through at least one waveform electrode as an applied waveform. At least one waveform electrode may be arranged in a waveform electrode array. The applied waveform may be received from the tissue of the subject through at least one return electrode, thereby completing an electrical circuit that includes the tissue of the subject as a component. In the various embodiments, electrical parameters of the applied waveform may be measured, including voltage, and phase, and electrical characteristics associated with the applied waveform may be calculated. Example electrical characteristics may include impedance of the tissue, reactance of the tissue, frequency response of the tissue, ratio of a change in impedance to a change in applied current, voltage, or other electrical parameter.

Complex impedance gradients exist in living tissue, and such impedance gradients may affect electrical measurements performed over the tissues, which are related at least in part to the cell membranes of the underlying tissues. It has been determined that electrode impedance exhibits inverse relationships to variable, increasing currents when studied at frequencies of less than or equal to about 10 kilohertz (kHz). Most living tissue is non-homogeneous and anisotropic; however, various embodiments are directed toward detection of tissues in non-homogeneous, anisotropic as well as homogeneous, isotropic tissue.

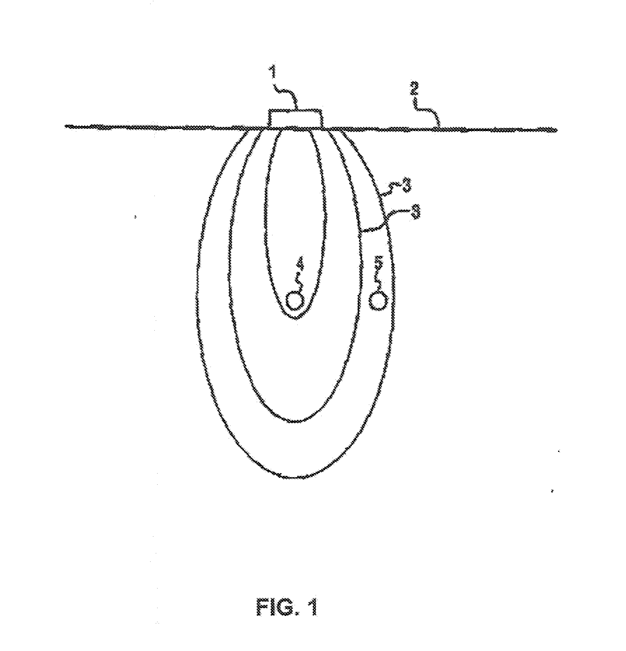

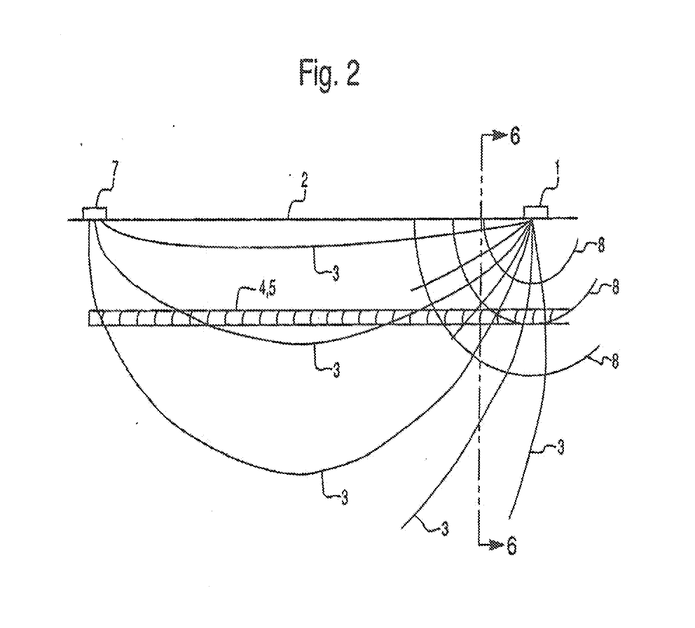

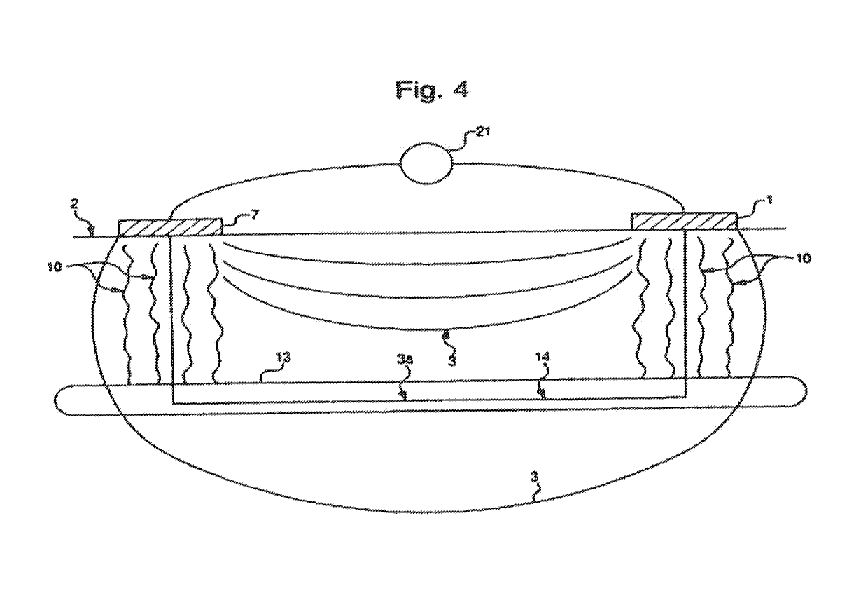

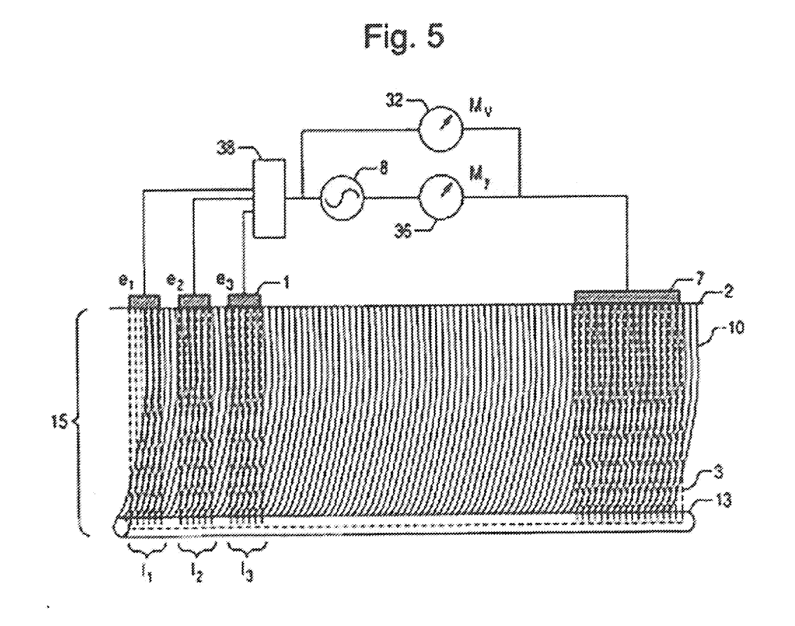

In an example embodiment illustrated in FIGS. 1 and 2, a waveform electrode 1 may be positioned on the skin surface 2 overlying ideal, homogeneous subcutaneous tissue in which reside biological, electrically responsive membrane systems (BERMS), such as nerves 4, 5. Nerves 4, 5 are shown as ideal, identical nerves, located the same distance beneath the skin surface, with nerve 4 at a normal angle to the position of waveform electrode 1 and the nerve 5 at an angle of less than 90° angle to waveform electrode 1. For an electrical field at 90° to the plane connecting the nerves 4, 5 and the waveform electrode 1 on skin surface 2, nerve 4 may experience a greater current density than nerve 5, for all applied current levels. As a result, the ratio of change in impedance to change in current (ΔZ/ΔI) may be greater for nerve 4 than for nerve 5. The shape of the current density distribution may be altered for actual, non-identical nerves, as discussed in further detail below with respect to FIGS. 4 and 5. However, current density may not be a critical factor in the various embodiments.

The scalar quantity current (i.e., electrical field strength) traditionally has been assumed to follow a spindle-shaped distribution between two skin surface electrodes 1 and 7 in homogeneous, conductive material. FIGS. 1 and 2 illustrate the current distribution in a homogeneous conductive medium. The current density at a point farther away from the center of the current distribution spindle may be lower than the current density closer to the center of the current distribution spindle. In a homogeneous medium, isocurrent lines 3 are formed in planes intersecting, at 90°, the line of the current-carrying electrodes. As shown, nerve 4 is located on an isocurrent line 3 having a higher current density than the isocurrent line on which nerve 5 is located. The actual current density at nerve 5 may be lower that at nerve 4 under these assumptions. As illustrated in FIG. 2, in the homogeneous portion of the medium, equipotential lines 8 are at right angles to the isocurrent lines 3.

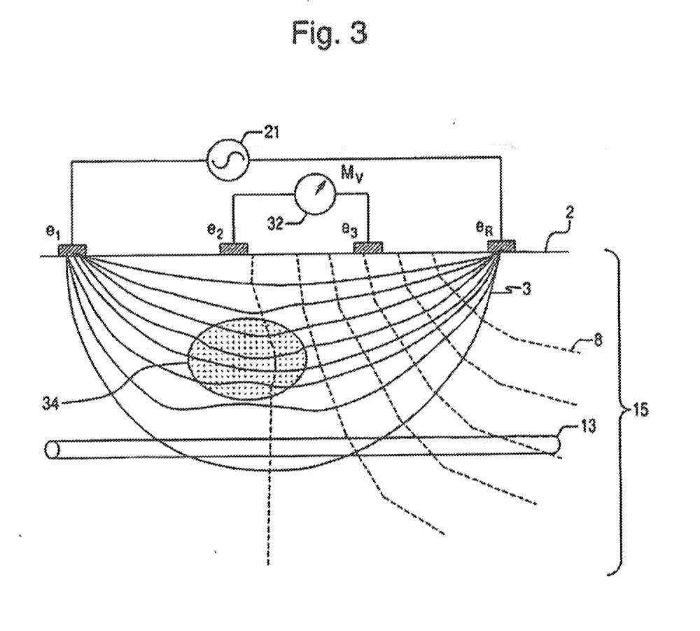

FIG. 3 illustrates an example of a non-homogenous tissue with portions of a subsurface structure 34 arrayed along an individual equipotential line 8. The portions of the subsurface structure 34 may experience different actual current densities depending on their distance from the center of the current distribution spindle. Thus, in a non-homogeneous medium, where tissue resistivity or susceptivity may be current, voltage, or frequency dependent, the resistivity or susceptivity of identical tissues may vary depending on the distance a measurement point lies from the center of the current distribution spindle. Alterations in applied current (I) or voltage (V) occurring at the skin surface 2 may cause the measured impedance (Z) at any point in the electrical field to change as a consequence of the resistivity or susceptivity variations induced by current density shifts at that particular measurement point. Electrical impedance tomography (EIT) is based upon this model of electrical field distribution through bulk tissue derived from theoretical current flow calculations for bulk conductors. The calculations utilized to process data gathered by EIT systems start with the application of Maxwell's equations in homogeneous, bulk conductors and modify the equations to account for non-homogeneities 34 within the bulk conductors, which represent tissues of varying resistivities.

Although complex back projection algorithms have been developed for use in EIT to create images of constituent tissues lying in an electrical field, the resolution of these images continues to be inadequate for routine clinical use. The inventors have determined that the underlying problem afflicting EIT back projection algorithms is that tissue is not only non-homogeneous, it is also anisotropic. The most remarkable anisotropic feature of living tissue is that the neuroanatomy represents preferential conductance pathways through tissue, altering current flow from a prolate ellipsoid shape to a more constrained and angular path following the major nerves. To provide a more valid model for EIT and FES (functional electrical stimulation) use, the nerve density and depth information beneath an electrode array assembly would have to be taken into account.

In contrast to EIT techniques, the various embodiments employ waveform electrodes of approximately 10 mm² or smaller to detect and distinguish the local variations in impedance on the skin which correspond to differences in the impedance of underlying tissue. In the various embodiments, an applied signal frequency may be in the range between approximately 100 Hertz and 10,000 Hertz. The calculated electrical characteristic may be impedance, although other electrical parameters may also be considered according to the various embodiments. To determine local impedance (or other electrical characteristic) at each electrode location, the various embodiments measure the local effect upon the signal applied between the waveform and return electrodes. A useful display of data from various embodiments may be a graph or tabular listing the measured impedance of each electrode in an array. Since the various embodiments rely on site-to-site differences, the underlying structures may be imaged without requiring a back calculation of field effects.

Determining locations of nerve tissue (i.e., tissue discrimination) may be affected by the concentration of voltage-gated channels in the cell membranes of biological tissues. Sodium and potassium channels act as voltage gated ion paths, so that in the presence of a transmembrane voltage gradient of sufficient magnitude for sufficient duration, the channel opens allowing a sodium or potassium ion to cross the cellular membrane. This movement of ions provides a preferential pathway for current flow through tissue. Nerve tissue is known to have the highest density of voltage-gated channels which, combined with its elongated structure, presents preferred conduction paths—indicated by low impedance—through tissue. The concentration of voltage-gated channels is lower in muscle, and even lower for other known cell types (e.g., endothelial cells in vessel walls).

Nerves are also known to resemble parallel conductors bundled together, wherein the resistance across the membrane (the transmembrane resistance) is greater than the resistance down the interior of the nerve (the longitudinal resistance). This structure facilitates conduction of electric fields down the axons of the nerve. The lipid bilayer structure of all cell membranes has a capacitance that has been consistently measured at around 1 microfarad per cm². The axons that comprise a nerve, with their long stretches of cylindrical cell membrane and, in many nerves, their multiple wrappings of Schwann cell membranes (the myelin sheath), comprise large capacitive structures. Since the axons of a nerve represent a parallel conductor, the total capacitance is the sum of the individual axonal capacitances. Consequently, resistance within nerves may be expected to be at a minimum compared to other tissues, while the capacitance of nerves may be expected to be at a maximum compared to other tissues. The relatively low internal resistance and large capacitance of the axons comprising nerves, compared to other tissues, may contribute to the ability to detect nerves according to the various embodiments. Further, it is believed that nerves exhibit a capacitive potential between individual axons isolated from each other and the body by connective tissue (i.e., epineurium, perineurium, and/or endoneurium). Axons also vary in diameter from about 6 microns to 30 microns. Consequently, the individual axons may demonstrate different capacitances based on both their length and diameter. The systems according to the various embodiments may fix the length variation through uniformly spacing the electrodes, thereby ensuring axon diameter as the primary effecter on individual axonal capacitance.

In the various embodiments, nerve tissue may be discriminated by identifying the (x, y) position of the nerve relative to a waveform electrode array, and by determining the depth position of the nerve using preferential pathways of the nerve, and impedance calculations in addition to the (x, y) determination. Further electrical characteristics and/or electrical state determinations may be used to contribute to the nerve discrimination in the various embodiments, for example, differential concentration, distribution, state (closed, inactive, or open) of voltage gated channels, as well the geometry and electronic properties of tissues, including the geometry (i.e., linear runs and branches) and electronic properties of nerves.

Living tissue is not only non-homogenous, but it is also anisotropic, and therefore electrical current may flow through tissues of a body along preferential pathways. Measurements indicate that preferential conductance pathways 10 from the skin surface 2 may be associated with the underlying neuroanatomy and directed at an approximately normal angle to the skin surface 2, as illustrated in FIGS. 4 and 5. Structures may be identified at the skin surface 2 that are associated with decreased impedances, which are at located at a normal angle to the plane of the skin surface 2 at those low impedance sites. An example structure is structure 13 in FIG. 4. Specifically, the preferential pathways presented by nerve tissue comprise a high density collector system in the dermal tissues leading into a long, uninterrupted, conduction pathway that is highly parallel and exhibits a large capacitance relative to non-nerve structures. Associated with this collector and conduction system is a right angle relationship from the skin surface to underlying nerve structures that is most likely a result of the anatomic relationships of nerves to the surrounding tissue. FIG. 5 illustrates brush-like, subcutaneous, dermal and epidermal axons 10 that extend from nerve 13 toward the skin surface 2, terminating short of the skin's outer surface, which may act as conductive pathways for this effect. The preferred conductive path presented by axons 10 and nerve tissue 13 results in electrical intensity lines 14 preferentially following an isocurrent path 3a through nerves 13 between electrodes 1 positioned on skin 2.

When a voltage is applied through the outermost epidermis layer (i.e., stratum corneum) of the skin surface 2 and the intervening subcutaneous tissue 15 between a waveform electrode 1 and a return electrode 7 current emitted by the waveform electrode 1 may flow down the brush-like structures of the dermal and epidermal axons 10 and then into the nerves 13. Current may flow along nerves 13, and then may pass back along axons 10 toward the skin surface 2 beneath the return electrode 7, flowing through the stratum corneum to be detected by the return electrode 7 in electrical contact with the skin surface. Individual axons may be modeled as leaky, one dimensional cables, which maintain the majority of the applied field intra-axonally, but allow some portion of the applied field to transit the surrounding tissue between axons or within a nerve bundle. Although the axoplasm demonstrates a bulk resistivity that is similar in magnitude to that of the extracellular fluid, the interior of axons lacks conduction barriers such as those presented by cell membranes in the surrounding tissue. An applied electrical field may travel in the extracellular fluid medium, but it may encounter these tissue barriers (represented as resistances and capacitances (RC) in series and in parallel) whereas the interior of the axon presents an ohmic resistance without the RC barriers.

Furthermore, there is a large capacitance associated with axon structure as a consequence of the long, cylindrical form of the axon, as described above. As the total number of axons within a nerve bundle increases, the total resistance is expected to fall asymptotically while the total capacitance progressively rises. Since impedance is directly related to resistance and inversely related to capacitance, the net result may be a large fall in impedance associated with nerve structures.

It is believed that the narrow zones of low impedance exhibited on the skin directly above nerves are due to the fact that axon fibers 10 preferentially rise from the nerve at approximately right angles to the skin surface and do not reach the skin at angles less than about 90 degrees. Thus, the low impedance zone due to the preferential conduction path through axon fibers appears just in the narrow zone of the skin that lies directly above the nerve. As such, in an embodiment, the presence and location of nerves may be revealed by localized zones (typically narrow lines) of low impedance measured on the skin. It has been found that in order to sense the local low impedance associated with an underlying nerve, the waveform electrodes may be constrained to a small area, preferably about 10 mm² or smaller. Larger electrodes, such as standard electro-cardiogram (ECG) electrodes which are typically square with sides of 1.5 cm or circular with diameters of 1.5 cm, and thus range in area from about 1.8 cm² to about 2.25 cm² (i.e., 180-225 mm²), electrically couple with the skin over areas much larger than the width of low impedance zones that lie above nerves, and thus determine average electrical characteristics of the skin (e.g., impedance) which washout the low impedance of an underlying nerve.

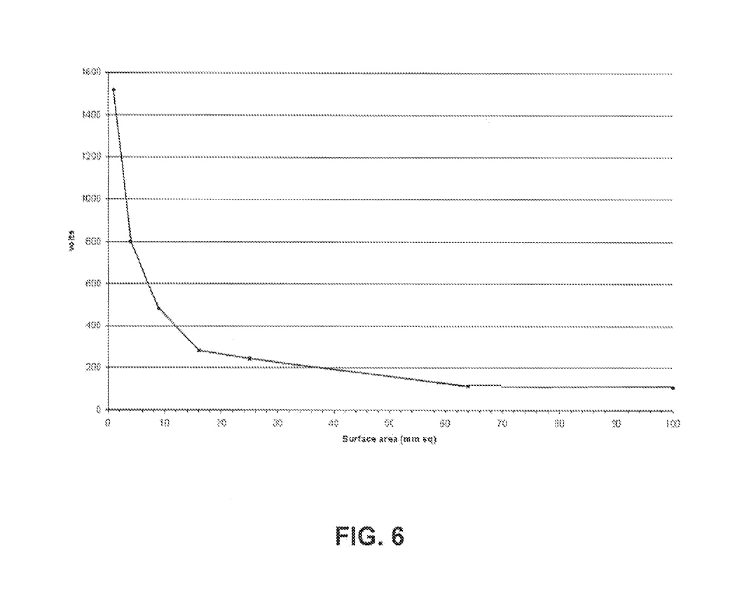

The effect of contact impedance and current density through the electrodes may affect the discrimination and location of nerve tissue according to the various embodiments. As the diameter of the electrodes decreases, impedance of the electrodes rises. FIG. 6 illustrates an example data set showing how changing surface area of electrocardiography (ECG) electrodes affects contact impedance, measured as applied voltage to maintain a constant current flow. In the study that yielded the illustrated example data, masked ECG electrodes were employed which involved placing two ECG electrodes together with a polyethylene mask in between. The masks had holes of variable diameter to simulate variable contact areas. The shape of the resulting data curve is shown to be a power function, and may be related to the area of the holes (πr²). With a smaller contact area (for example, less than 10 mm³), contact impedance may become a progressively more significant source of measurement error.

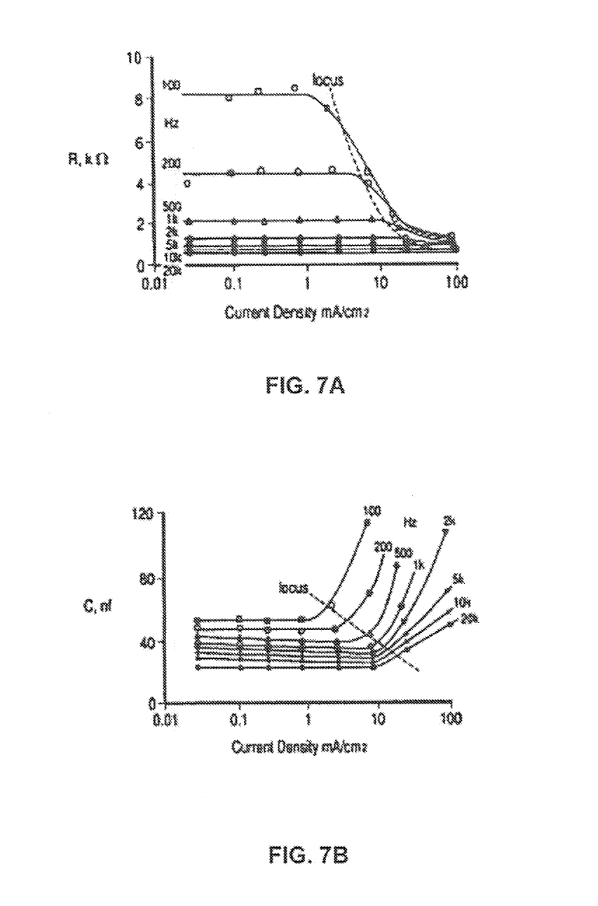

As illustrated in FIG. 7A, resistance of signals through electrodes becomes non-linear as the current density of signals increases and frequency decreases. As illustrated in FIG. 7B, capacitance of signals through electrodes also becomes non-linear as the current density of signals increases and frequency decreases. These electrical characteristics suggest that in order to maintain stable electrode impedance for skin surface measurements, the current density should be kept within the linear ranges for current density and frequency, which may be dependent on the electrode material. A suitable range of current densities for use in various embodiments may be from approximately zero mA/cm² to approximately 10 mA/cm², more preferably from approximately 0.2 mA/cm² to approximately 10 mA/cm².

The graphs shown in FIGS. 7A and 7B were determined from stainless steel electrodes. The main consideration for a 3 mm diameter electrode was found to be the contact impedance, which is a power function. As the diameter of the electrode decreases, impedance rises, with a knee of the curve being around 10 mm2. With electrode contact areas less than 10 mm2, contact impedance becomes a progressively more significant source of measurement error. So, both current density and contact impedance considerations may play a role in electrode diameter selection.

Therefore, both contact impedance and current density are factors to be considered in selection of the electrode size. By way of example but not by way of limitation, an electrode approximately 5 mm in diameter has an area of approximately 0.1 cm². Balancing the various characteristics against the aim of detecting localized impedance differences may lead, for example, to a suitable range for the diameter of electrodes used with various embodiments may be between approximately 1 mm and approximately 6 mm, more preferably between approximately 2 mm and approximately 5 mm, and even more preferably approximately 3 mm in diameter. Such electrodes may have an area of approximately 10 mm² or less. Further, a suitable range of currents used in various embodiments may extend between approximately 10 μA and approximately 600 μA, more preferably between approximately 10 μA and 400 μA and even more preferably between 10 μA and 100 μA.

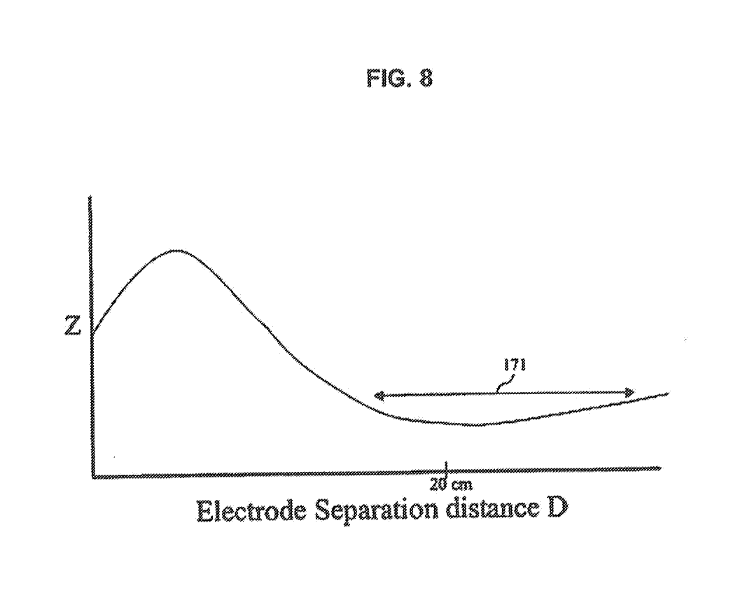

In the various embodiments, for a given set of measurement conditions, a distance may exist between the waveform electrode 1 and the return electrode 7 over which impedance is at a minimum and nerves 13 may be discriminated by observing changes in impedance with waveform electrode 1 and return electrode 7 at such spacing. FIG. 8 illustrates impedance measurements that correspond to changes in separation distances between the waveform electrodes and return electrode. Over short separation distances, the measured impedance rises to a maximum. Beyond the maximum, the impedance declines asymptotically toward non-zero minimum value and then trends upwards approximately linearly. Observations have determined that better (e.g., more revealing) nerve identification is obtained with separation distances in the tail region 171 of this Z vs. D curve. For example, about 20 cm may be a workable separation distance. In the tail region 171, the rate of change of impedance with distance is lower, so that reducing the difference between the first and last rows in the array has less effect than at shorter separation distances. The optimum separation distance may vary based upon the individual, the body portion being examined, etc. For example, for pediatric subjects, the optimum separation distance may be different than for adult subjects. Thus, a method of applying electrodes to a subject may include placing the waveform electrode 1 and return electrode 7 at a proper distance apart to facilitate obtaining better data. For example, the waveform electrode 1 and return electrode 7 may be positioned in the range approximately 20 cm apart.

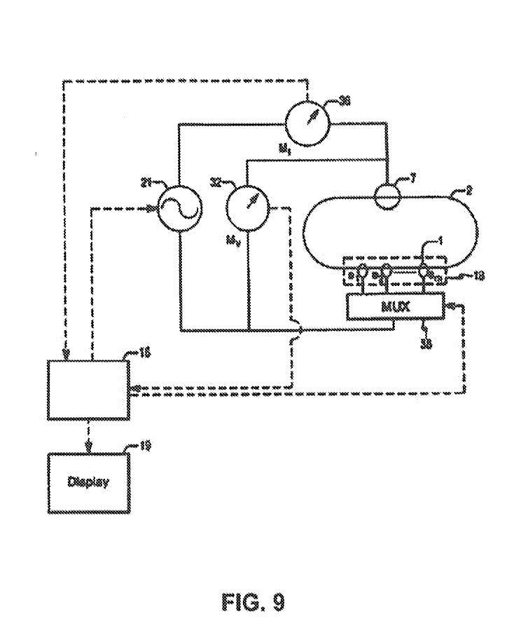

FIG. 9 illustrates a tissue discrimination system that may be used to locate nerves according to an embodiment. The controller 16, such as a microcomputer, microcontroller or microprocessor, may be configured to control the generation of electronic signals, receive detected signals, store data, and perform analysis of the received data; a waveform generator 21, an electrical property measuring sensor (e.g., voltage meter 32 and/or current sensor 36); one or more waveform electrodes 1; and a return electrode 7. The waveform electrode 1 may be a plurality of waveform electrodes E₁ . . . Eₘ, e.g., configured in the form of an electrode array assembly 18, to which the signal generator 21 may apply input signals. In an example embodiment, waveform electrodes 1 may be about 10 mm² in area or smaller in order to permit them to determine localized variations in impedance. In an embodiment, a multiplexer switch 38 may be used to switch the waveform input signals to specific electrodes 1. The effects on the applied waveform of the tissue underlying the skin 2 on which the electrodes 1, 7 sit may be sensed by a sensor between the waveform electrode 1 and the return electrode 7. The sensor may be any of a number of electrical signal sensors known in the art, such as a voltage measuring device 32 and/or a current measuring device 36. The sensor provides measurement data signals to the controller 16 for analysis. In alternative embodiments, the waveform electrode 1 to which input signals are applied may be a single electrode, while the return electrode 7 may be one of an electrode array assembly to sense the resulting signal. The system may also include a display 19 electronically coupled to and configured to receive display signals from the controller 16 and to generate a visual display of results, e.g., data displays in configurations that enable a user to detect or see the presence of nerves within the subject. In an embodiment, the at least one waveform electrode may be a plurality of waveform electrodes and the at least one return electrode may be a plurality of return electrodes.

In the various embodiments, electrodes may be placed in electrical contact with the skin of a subject. A controlled current or voltage may be applied, and may be measured through the tissue from the same electrodes, from which electrical characteristics or properties of the tissue, such as impedance, may be calculated. In an example embodiment, a controlled current may be applied to the electrodes, and the voltage between the electrodes may be the electrical property measured. In an alternative example embodiment, a controlled voltage may be applied to the electrodes, and the current through the underlying tissue may be determined, from which impedance may be calculated. As is well known in the art, electrodes may be placed in electrical contact with skin by placing the electrode in physical contact with the skin, preferably with a coupling interface material 31, e.g., a hydrophilic, silver-silver chloride gel. In the various embodiments, comparing the received signal to the applied signal may provide information on the electrical characteristics (e.g., impedance or admittance) of tissue between the waveform electrode 1 and return electrode 7.

A waveform of the applied signal may be any wave shape, and more preferably may be either of a monophasic or a biphasic sinusoidal or square wave form. The waveform may be a sinusoidal wave, a rectangular wave, some other periodic wave, a constant non-zero amplitude waveform, a single impulse, some other aperiodic waveform, or some additive combination thereof. One preferred waveform (herein referred to as a monophasic sinusoidal waveform) is the combination of a sinusoidal waveform plus a constant offset level resulting in entirely non-negative current or voltage amplitudes throughout the waveform. The frequency of a time-varying applied signal may range from approximately 1 Hz to approximately 10 kHz, more preferably between approximately 0.1 kHz and approximately 5 kHz.

In the controlled current mode, measurements of the sensed signal may be made immediately upon applying the source signal, after approximately 100 cycles (or more), or at any time in between, more preferably after approximately 20 cycles. A tissue charging effect is observed when using a controlled current waveform, necessitating about 50-70 cycles to complete the charging effect. The tissue charging effect is not observed using controlled voltage, since current is allowed to “float” and charging may occur within 2-5 cycles. As such, in the controlled voltage mode, measurements of the sensed signal may be made immediately upon applying the source signal, after approximately 100 cycles (or more), or at any time in between, and more preferably after approximately two to approximately five cycles.

In an embodiment, the impedance (Z) may be determined for all the electrodes in the array. Electrodes with the lowest Z value will overlie the course of the nerve structure most directly, or have the largest quantity of nerve tissue (e.g., a nerve branch point) underlying those electrodes. In an embodiment, the resistive (R) and reactive (X) components of Z may be derived, noting that the electrodes demonstrating the lowest resistivity, or highest capacitance, will most directly overlie the course of the nerve structure. Other, derivative functions of current or voltage related to frequency, time, or distance may also be used to indicate the position of nerve structures.

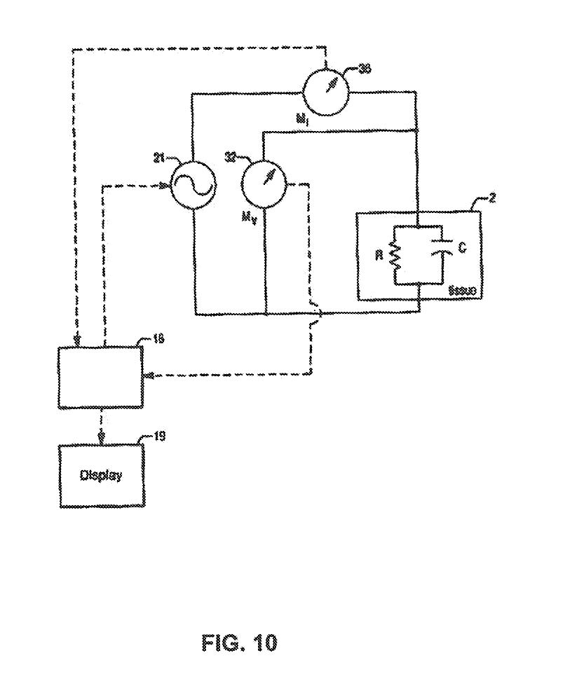

Tissue discrimination by the system of the various embodiments may rely on the resistor-capacitor circuit characteristics of tissues. FIG. 10 illustrates that the effects on the applied waveform of tissue electrical characteristics may be modeled as a parallel RC circuit element. The impedances corresponding to each electrode in the array, which correspond with underlying tissue structures, may be selectively determined for each generated waveform. After determining impedance values, various mathematical analyses may be performed using the plurality of impedance determinations, including coordinate determination of a nerve's location in three-dimensional space. The various embodiments may include other mathematical analyses, for example, determining a ratio of impedance change to the applied current change. The mathematical analyses may also consist of calculations to support any effective data presentation technique, including but not limited to presentation of: raw data, normalization of raw data, rates of change between neighboring electrodes, rolling averages, percentage difference, of derivative functions, or more complex analyses e.g., Fourier analysis of frequency components, all of which may be presented graphically and/or numerically such as in a table of values.

The controller 16 may also determine from measured data the individual components of the impedance measurement, namely the resistance (R) and reactance (X). These may be calculated using known means, e.g., using a Fourier analysis technique to obtain the real (resistive) and imaginary (reactance) components of the impedance. Similarly, the controller 16 may calculate other electrical characteristics, such as permittivity, inductance, capacitance, etc.

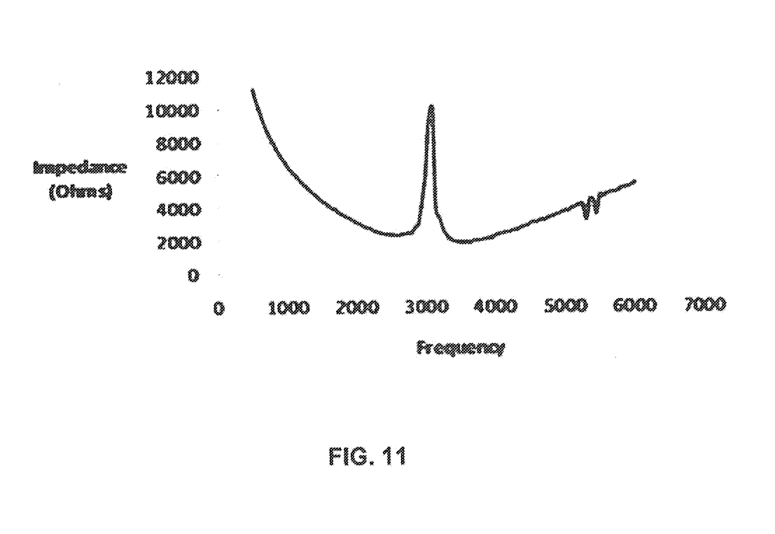

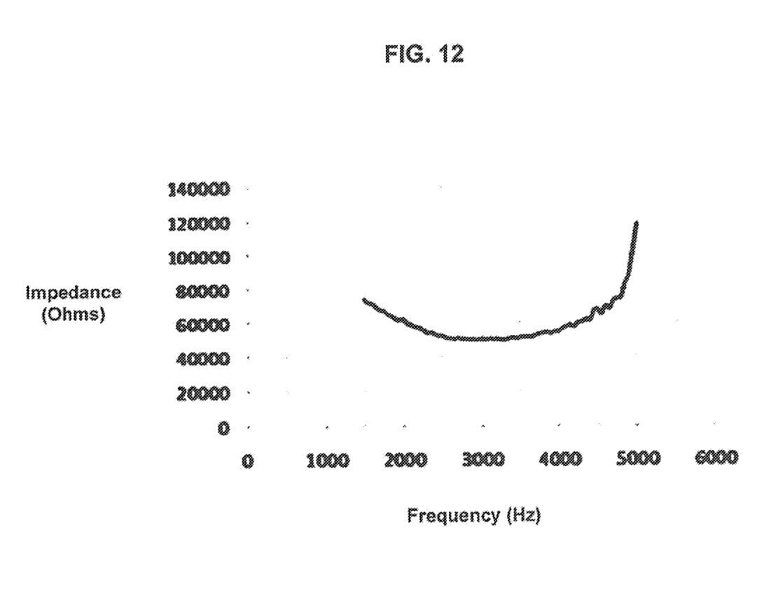

The inventors have discovered that although no true inductance is present in living tissue, the circuitry of the measuring system according to the various embodiments does demonstrate inductance. FIG. 11 illustrates impedance measurements at various frequencies over tissue containing high densities of nerve tissue, showing that, over tissue containing high densities of nerve tissue, this circuitry-related inductance is sufficient to cause electrical resonance phenomenon. FIG. 12 illustrates impedance measurements at various frequencies over tissue containing low densities of nerve tissue, showing that no resonance occurs over tissue containing low densities of nerve tissue.

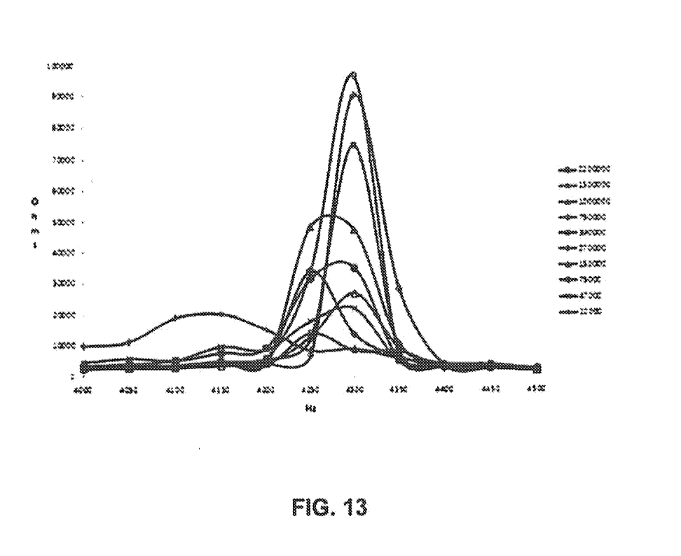

Additionally, FIG. 13 illustrates of impedances calculated at a fixed frequency over a multiplicity of RC circuits, all having capacitances of 1×¹⁰⁻⁸ farads (the range of nerve-related capacitance) and differing resistances. In an embodiment, combinations of these RC circuits may result in a composite decay curve with variable characteristics similar to tissue, depending on the constituent RC circuits.

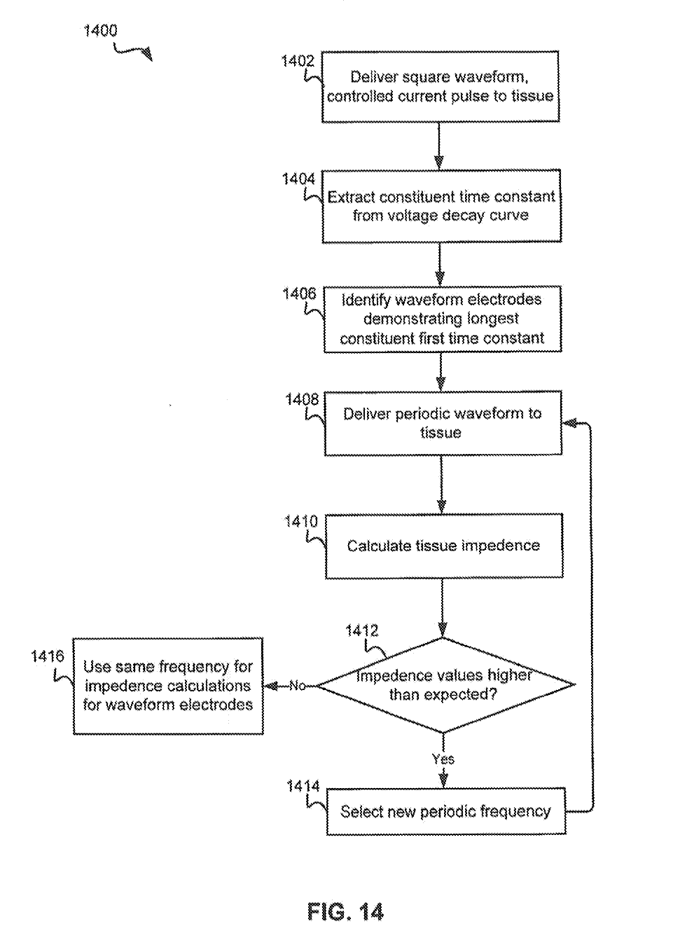

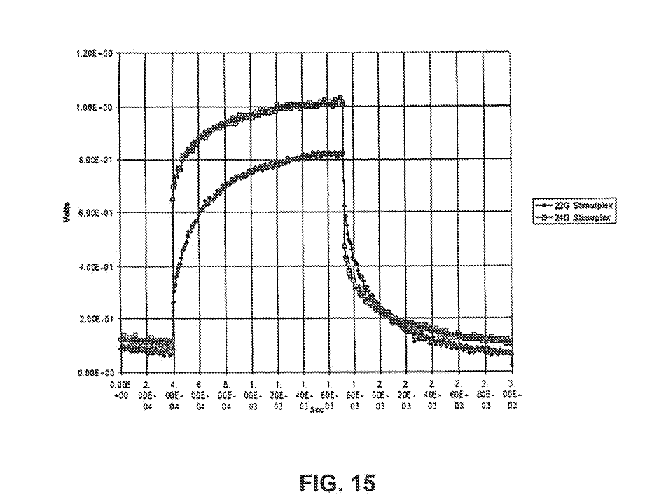

The electrical resonance caused by circuitry-related inductance may cause impedance values to be larger than anticipated when determined over high density nerve tissue regions, which may result in loss of nerve discrimination. FIG. 14 illustrates an embodiment method for detecting the effect of electrical resonance on impedance values by the use of time-constant determinations, and using different frequency, periodic waveforms to determine a frequency that avoids resonance. In method 1400, a single square waveform, controlled current pulse may be delivered to tissue, step 1402, which results in a voltage decay curve showing an exponential form, illustrated in FIG. 15. This observed voltage decay curve observed voltage decay curve represents a composite of individual RC time constants derived from the various tissues in the current path, according to the relationship:

V=ΣC₀e^{-t/τ}^{₀}+C₁e^{-t/τ}^{₁}+C₂e^{-t/τ}^{₂}+ . . . +Cₙe^{-t/τ}^{ₙ}

Where V is voltage, t is time in seconds, i is the RC time constant, and Cₙ₋₀ are mathematical constants unique to each RC circuit. The tissue RC circuit that has the longest time constant dominates the form of the voltage decay curve seen with a controlled current square waveform pulse. Since the resistivities of the tissues involved in the electrical path are similar in magnitude, capacitance plays the largest role in determining the time constant. Nerve tissue has the highest capacitance of the tissues in the electrical path, and as a consequence, has the longest time constant. In step 1404, the constituent time constants may be extracted from the voltage decay curve, for example, using the logarithmic stripping method described by Rall (Rall, Time Constants and Electronic Length of Membrane Cylinders and Neurons (1969); see also Rall, Membrane Potential Transients And Membrane Time Constant of Motoneurons (1960)). In this manner, waveform electrodes demonstrating the longest constituent first time constant may be identified, step 1406.

In step 1408, a sinusoidal waveform may be delivered to the tissue. Impedance may be calculated for each of the identified waveform electrodes in step 1410. The lowest calculated impedance values may be compared with the first time constant extracted for the identified waveform electrodes to identify an acceptable frequency for impedance determination, determination 1412. If the comparison demonstrates unexpectedly high impedance values (determination 1412=“Yes”), electrical resonance is indicated. A new sinusoidal frequency may be chosen for the applied electrical field used to determine impedance (e.g., by prompting the operator to select another frequency), step 1414. Steps 1408-1414 may be repeated for the waveform at the new frequency, until the waveform electrodes that have the longest first constituent times do not demonstrate unexpectedly high impedance values (determination 1412=“No”), showing a match-up between the lowest impedance readings and the longest time constants and therefore indicating an acceptable frequency. The acceptable frequency may then be used to calculate impedance for all waveform electrodes, step 1416. Once impedance is calculated at an acceptable frequency for all of the waveform electrodes, the electrode having the lowest impedance may be properly identified.

In areas where the outermost layer of skin is broken (i.e., from a scratch or puncture), impedance is lost. In an alternative embodiment, the determined time constants may be used to discriminate between low impedance sites that are related to the position of the nerves, and those that are caused by skin breaks. In this manner, impedance neurography may be performed without the need for intact skin. Some examples of this application may include identifying nerves during surgery.

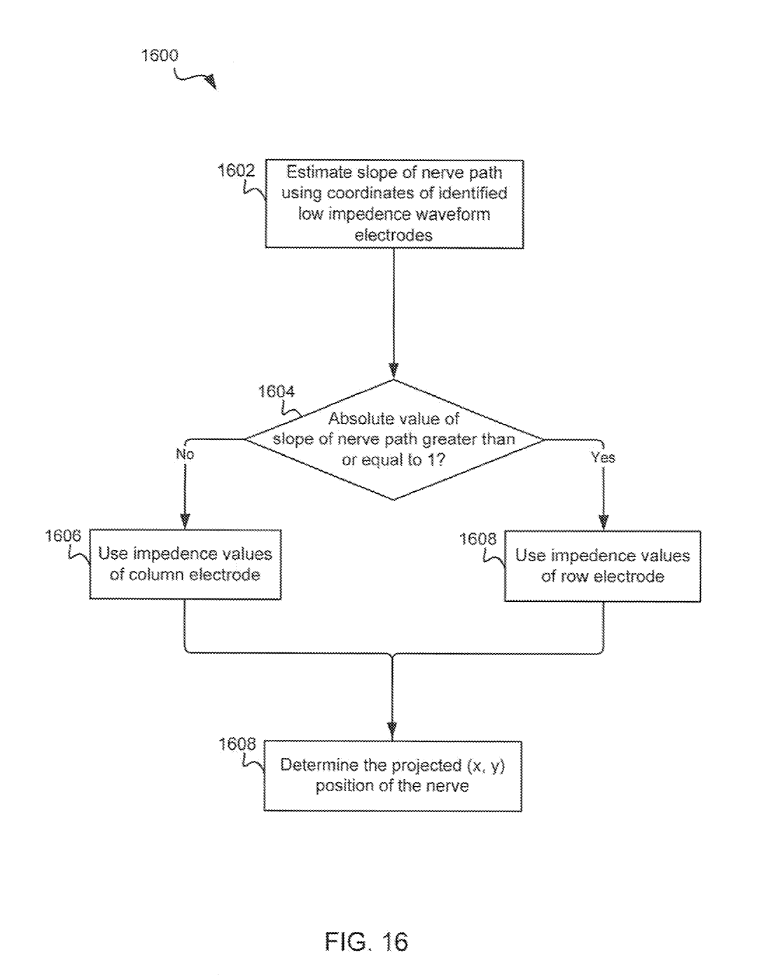

Once the waveform electrodes demonstrating the lowest impedance are identified, the interpolated position of a nerve may be determined. FIG. 16 illustrates a method of determining a projected (x, y) position of the nerve in the two-dimensional field of the waveform electrode array. In method 1600, an estimate slope of the nerve path relative to the waveform electrode x, y grid may be determined using coordinates of the identified low impedance waveform electrodes, step 1602. For example, the positions of the lowest-impedance electrodes (A₁ and A₂) may be represented as (x_{A1}, y_{A1}) and (x_{A2}, y_{A2}), respectively. Therefore, the estimated slope M may be determined according to the equation:

$$M = \frac{( {y}_{A 2} - {y}_{A 1} )}{( {x}_{A 2} - {x}_{A 1} )} .$$

In determination 1604, if the absolute value of the slope of the nerve path is greater than, or equal to 1 (i.e., determination 1604=“Yes”), impedance values from the rows of waveform electrodes in the waveform electrode array may be used to determine the nerve position, step 1606. This is because a more vertical slope reveals the nerve path is to be crossing several rows of waveform electrodes. If the absolute value of the slope of the nerve path is less than 1 (i.e., determination 1604=“No”), impedance values from the columns of waveform electrodes in the waveform electrode array are used to determine the nerve position, step 1608. This is because a more horizontal slope reveals that the nerve path crosses several columns of waveform electrodes. Using row or column values, the projected (x, y) position of the nerve may be determined.

In an example embodiment, a projected (x, y) position of a nerve interpolated between row waveform electrodes may be determined based on equations and relationships described below. The lowest impedance (Zₘᵢₙ) from the total waveform electrode array may be identified, and assumed to represent the impedance value at a point on the skin surface lying closest to a normal to the underlying nerve.

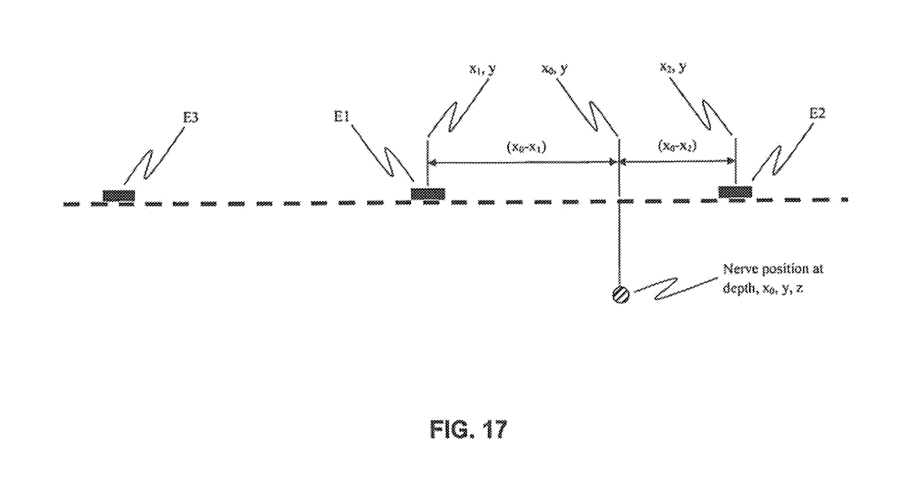

FIG. 17 illustrates an example of the coordinates for determining the projected (x, y) position of the nerve using impedance values from the rows of waveform electrodes. The lowest impedance waveform electrode in a given row may be identified (E₁), with an impedance value of Z₁. The impedance values from the immediately adjacent row waveform electrodes (E₂ and E₃) may be determined, and set as Z₂ and Z₃. Since the nerve position is between the two lowest waveform electrodes, their impedances may be selected for interpolation (Z₁ and Z₂). Thus, as illustrated in the representation of the waveform electrode array 902, the position of the projected nerve is (x₀, y), the position of E₁ is (x₁, y), and the position of E₂ is (x₂, y). As also illustrated in the example representation of the waveform electrode array 902, x₁=0. Therefore, the inter-electrode spacing between E₁ and E₂ may be considered x₂. The x-axis distance between the position of the projected nerve and E₁ may be considered (x₀−0) and the x-axis distance between the position of the projected nerve and E₂ may be considered (x₂−0). The impedance differences between the position of the projected nerve and E1 may be considered Z₁ or ΔZ₁. The impedance differences between the position of the projected nerve and E2 may be considered Z₂−Zₘᵢₙ, or ΔZ₂. The relationship describing the projected nerve position, in the x-axis is:

$$\frac{( {x}_{0} - 0 )}{Δ {Z}_{1}} = \frac{( {x}_{2} - {x}_{0} )}{Δ {Z}_{2}}$$

$${x}_{0} ( \frac{Δ {Z}_{2}}{Δ {Z}_{1}} ) = {x}_{2} - {x}_{0}$$

$${x}_{0} ( 1 + \frac{Δ {Z}_{2}}{Δ {Z}_{1}} ) = {x}_{2}$$

$${x}_{0} = \frac{{x}_{2}}{( 1 + \frac{Δ {Z}_{2}}{Δ {Z}_{1}} )}$$

Using the determined slope of the nerve path, the value for y may be determined. The relationship describing the projected nerve position in the y axis when using column data is analogous.

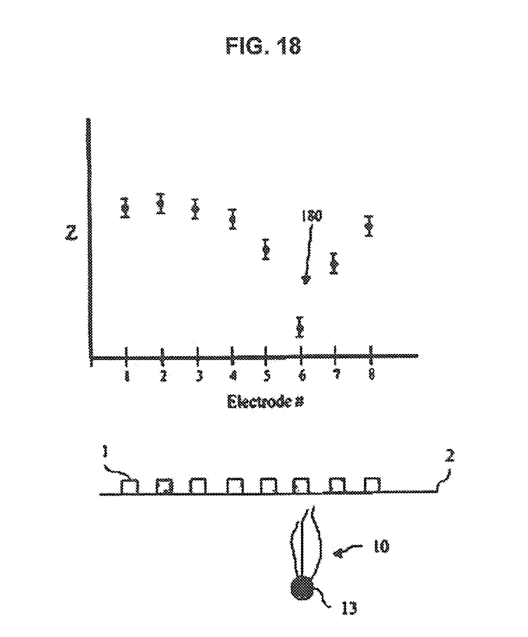

As illustrated in FIG. 18, dermally projecting axons 10 have been found to extend from nerves 13 toward the skin 2 only at a right angle to the skin 2, thereby providing a preferential conduction path at right angles to the skin 2. The right-angle relationship exists between the position of high density nerve tissue and the lowest impedance point at the skin surface; that is, the low impedance site lies on a normal between the complex curve of the skin surface and the position of the nerve at depth.

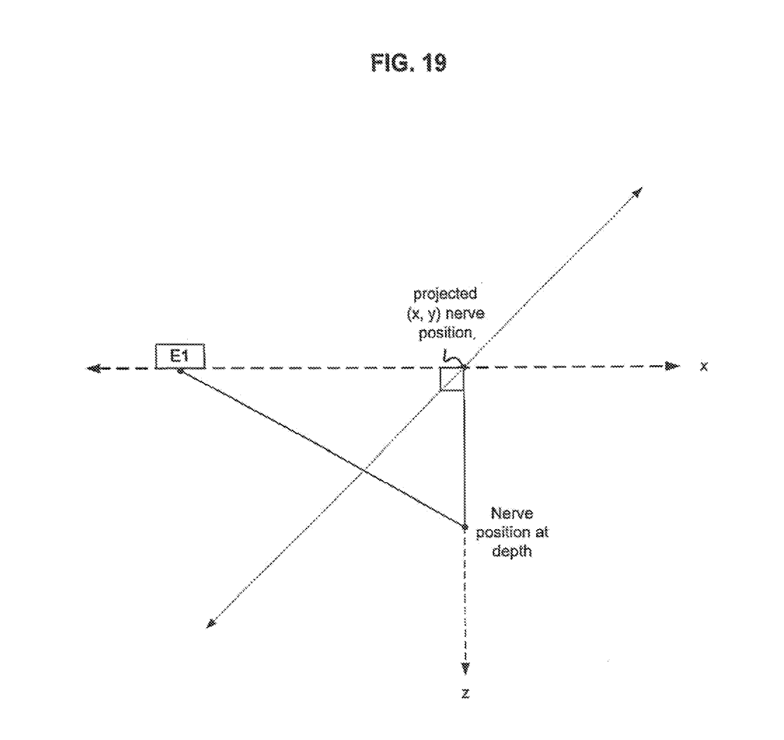

FIG. 19 illustrates a theoretical right triangle that may be represented in the z-plane to determine the nerve depth, according to an embodiment. A first apex of this triangle may be the projected (x, y) position of the nerve determined as described above with respect to FIG. 17. A second apex of this triangle may be the position of the lowest impedance waveform electrode E₁. The distance between the waveform electrode position E₁ on the skin surface and the projected (x, y) position of the nerve may form the base of the right triangle. This relationship appears to be related to the embryology of nerve development where larger nerves run parallel to the skin surface, at depth, and perforating branches run normal to the skin surface. Combined with the observation that nerves are preferential current pathways, i.e., anisotropicities, it is implied that use of a bulk conductor model for current flow does not accurately define nerve depth, which has been shown experimentally.

In an embodiment, the depth of the nerve may be determined using the projected (x, y) position of the nerve as described above with respect to FIG. 17, the theoretical right triangle described above with respect to FIG. 19, and the mathematical relationship of impedance to length for an electrical path to the nerve using known equivalent circuit models. This determination of depth assumes a constant of proportionality that relates impedance to the length calculations, in other words that the ratio of impedance to length calculations over all the electrodes should equal a constant.

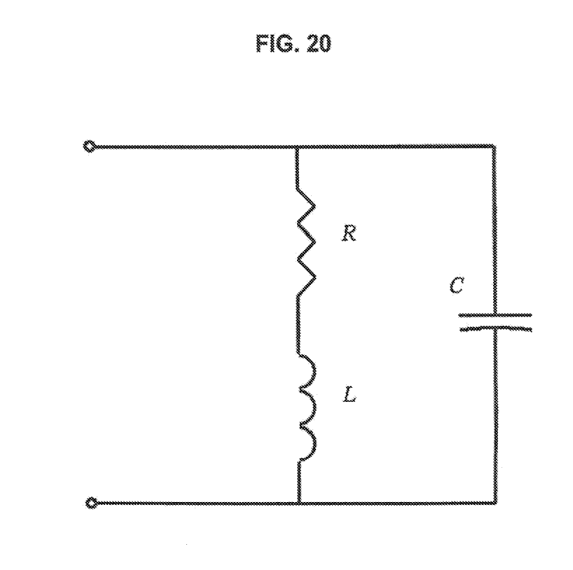

FIG. 20 illustrates an equivalent RLC circuit that may be used to determine the relationship of impedance (Z) to length (l), recognizing that resistance, capacitance, and impedance are directly related to the length of the nerve (or, for example, the length squared). In circuit 1102, the impedance of the resistor and inductor in series may be represented as Z_{R--L}=(\sqrt{R²+X_{L}²}, or Z²=R²+X_{L}², wherein R is resistance and X_{L} is reactance of the inductor. The impedance of the capacitor in parallel with the resistor and inductor may be represented as

$$\frac{1}{{Z}_{RLC}^{2}} = ( \frac{1}{{R}^{2} + {X}_{L}^{2}} ) + \frac{1}{{X}_{C}^{2}} ,$$

wherein X_{C} is the reactance of the capacitance. Therefore:

$$\frac{1}{{Z}_{RLC}^{2}} ∝ ( \frac{1}{{l}^{2} + {l}^{2}} ) + \frac{1}{\frac{1}{{l}^{2}}} , or \frac{1}{2 {l}^{2}} + {l}^{2} , \\ and \frac{1}{{Z}_{RLC}^{2}} ∝ ( \frac{1 + 2 {l}^{2}}{2 {l}^{2}} ) , \\ therefore , \frac{1}{{Z}_{RLC}} ∝ {( \frac{1 + 2 {l}^{4}}{2 {l}^{2}} )}^{- 0.5} .$$

In the right triangle, due to the anisotropicities discussed above, the length of the electrical path to the nerve may be calculated as the sum of the first non-hypotenuse leg and the second non-hypotenuse leg. The first non-hypotenuse leg may be represented as the distance from the position of E₁ and the projected (x, y) position of the nerve, and the second non-hypotenuse leg is equal to the nerve depth. Thus, using the determined relationship of impedance to length, a table may be generated that correlates impedance values with nerve depth values. Thus, a nerve depth value may be determined that corresponds to the measured lowest impedance value for the array. The calculation of depth assumes proportionality that relates impedance to the length calculations, i.e., the ratio of impedance to length calculations over all the electrodes should equal the constant.

A number of parameters may be measured or calculated and used for various nerve imaging and diagnostic purposes in combination with the nerve location determinations of the various embodiments. For example the maximum signal level measured after the applied signal has been applied for sufficient time for the average measured signal to reach an approximately steady state may be calculated. This maximum signal may be used to determine or estimate a number of characteristics of the nerves underlying the skin, including by way of example but not by way of limitation: the size of the underlying nerve; a relative indication of nerve health and/or function; nerve injury; the depth or distance of the nerve from the electrode; and the presence or absence of major nerves in the vicinity of the electrode. Additionally, the maximum signal level may be used to calibrate or contrast the efficiency of various electrodes, e.g., to detect an electrode with poor electrical coupling to the skin. The magnitude of the difference between peaks and valleys may indicate the relative admittance of the underlying tissue, including in particular nerves in the vicinity of the electrode. The difference as a measure of relative admittance may be used to determine or estimate a number of characteristics of the nerves underlying the skin, including by way of example but not by way of limitation: nerve activity, nerve health and/or function, the depth or distance of the nerve from the electrode, and the presence or absence of major nerves in the vicinity of the electrode. Other useful features may include the time to reach the maximum signal level and the phase shift between the signal and the measured data. Further, the change in the measured signal after an applied waveform is terminated may also yield important information about the underlying tissue, such as the rate of decay of the bias signal and the time for the signal to return to zero. These various parameters may be used singularly or in combination with one or more other characteristics to distinguish tissues, for nerve imaging and/or for nerve diagnostic purposes. Further, the collection of measurement data and the calculations may be performed by automated systems that may translate the various electrical characteristics of the measured signal to deduce information about the tissue underlying the electrodes.

Another application of the various embodiments may build on findings of Brown, et al. in Blood Flow Imaging Using Electrical Impedance Tomography, (Clin. Phys. Physiol. Meas. 1992; 13 suppl A: 175-9) discussing the use of real time electrical impedance tomography (EIT) to discern the flow of blood through the vascular system. By combining the techniques of EIT, which may discern blood flow, with nerve imaging according to the various embodiments, both blood vessels and nerves may be distinguished using the same electrode array assembly to provide a more complete depiction of the underlying neurovascular anatomy. Thus, an embodiment combines EIT with tissue discrimination data according to an embodiment to yield information based both on intracellular and extracellular conductive paths and phenomenon. Such combination of EIT and tissue discrimination according to an embodiment may be accomplished by conducting both scans using the same electrode array assembly or by using data registration to permit two-dimensional or three-dimensional correlation of data from the two technologies to yield a combined image.

In another application, the various embodiment methods may be combined with EIT technology and/or other imaging technologies based on different physical phenomena, such as X-ray (e.g., a CT scan), magnetic resonance imaging (MRI), positron emission tomography (PET), and ultrasound. It is expected that combining imaging results from different physical phenomena, which interact with tissue in different ways, may provide improved discrimination and resolution of tissues compared to any single imaging technology. This embodiment may be particularly useful in identifying and locating breast cancer tumors where the different phenomenological imaging technologies may be combined to more clearly discriminate tumor from healthy tissue.

As shown in the various embodiments, preferential conduction paths exist at right angles to the skin for underlying nerves. As discussed above with respect to FIG. 19, the electrode directly over a nerve may exhibit measurably lower impedance Z compared to adjacent electrodes even though the path length from adjacent electrodes to the nerve 13 is not significantly different. This characteristic of nerve axons may be used to simplify nerve discrimination and location when the electrodes are constrained in area (e.g., <approximately 10 mm²). The crossing point of a nerve and a row in the electrode array assembly may be found by identifying the electrode exhibiting the lowest impedance (or highest permittivity, highest conductivity, etc.). By aligning results for each of the rows in the array on a display, the path of a nerve may be traced from valley to valley for impedance (or peak to peak for characteristics like permittivity and conductivity) across the array. Such an analysis may be readily accomplished visually by displaying a matrix of values, or calculated using Microsoft Excel® or similar software, though more sophisticated analysis software is preferred.

For the purposes of providing an easy to understand display, the minimum and maximum of a display of the measured admittance or voltage may be scaled to an arbitrary range, e.g., from 0 to 1 or from 0 to 100%, or any other scale. Normalizing data for display may also be accomplished with colors or shaded displays where electrode locations or areas featuring relatively stronger signals are indicated with lighter colors or shades compared to electrode locations or areas featuring relatively weaker signals. Using color (or gray-scale) displays to indicate received signal magnitude, recorded signal values from all electrodes may be presented in a 2-D display 190. Instead of presenting this display on a computer terminal, an alternative configuration comprises small illuminators, e.g., LEDs, positioned on the top surface of each electrode in the array to provide a direct indication of the underlying nerve. Indicating measured signal strength with a relative luminosity of each illuminator may provide a simple yet effective display of underlying tissue. For example, if a relatively strong signal is indicated with a relatively light illuminator, a moderate signal is indicated with a moderately dim illuminator and a weak signal indicated with a dim illuminator, the path of the underlying tissue driving the signal may be viewed directly.

Electrical characteristics of the tissues between waveform and return electrodes may be derived from the measure electrical parameters by a number of methods different methods and algorithms. The following examples analyze a series of discrete measurements made at discrete times t to estimate coefficients of a mathematical function F(t) which provides a “best fit” approximation match to the digital numeric sequence W′ at time t=iT, where T is the interval between discrete measurements i and W′ is the sequence of measurement values. Such a mathematical function may be chosen from a number of parameterized functions that differ only by the values of a small number of parameters or coefficients. The independent variable of the function may be time t or a unit related to time (e.g., clock cycles, sample numbers, etc.).

Such a mathematical function may be a composite (sum or product) of several simpler component or basis functions with the same independent variable t. These component functions may be, for example, a constant amplitude value; one or more periodic (cyclic) functions such as a conventional sine function or cosine function, square wave; and/or an exponential decay function asymptotic to zero. These component functions may be consistent with the electrical characteristics expected of a parallel RC circuit. For example, a constant amplitude value may reflect an offset (e.g., direct current) component of an applied waveform, the cyclic function reflects the cyclic nature of the applied waveform, and the decay function may reflect the capacitive nature of tissue, including nerve tissue, in the presence of an electric field. Thus, this embodiment may involve estimating specific parameters of the terms of such a composite mathematical function so that the resulting function approximates (i.e., forms a “best fit” approximation for) the sequence of digital values at the times associated with those values. For example, a suitable mathematical function may be:

$$F ( t ) = {A}_{D C} + {A}_{A C} cos ( \frac{2 π T}{NT} + {P}_{0} ) + {A}_{0} {e}^{- {A}_{RC} T}$$

where
A_{DC} is the amplitude of the constant direct current component,
A_{AC} is the amplitude of the periodic component,
A₀ is the amplitude of the decay component,
A_{RC} is the decay rate constant,
T is the interval between discrete measurements, and
N is the number of samples per cycle.

Using the parametric constants that characterize the best fit mathematical function F₍ₜ₎ the electrical properties of the tissue may be derived. The electrical properties of interest may include any of impedance, admittance, resistance, susceptance, capacitance, or phase shift, for example. Regardless whether the voltage or the current is the controlled property of the electrical waveform applied to the tissue, when using a sinusoidal waveform the complex form of Ohm's Law may be applied to find the complex impedance Z of the tissue by Z=V/I, where Z is the impedance (with real resistive and imaginary reactive components), V is the periodic component of the voltage waveform (either applied or measured), and I is the periodic component of the current waveform (either applied or measured), and where complex quantities V and I are measured with respect to the same phase reference (i.e., synchronous cosine and sine references). If it is assumed that the parameters A_{AC} and P₀ are known for each of the component functions approximating cyclic voltage waveform and current waveform, then the complex impedance Z is:

$$Z = ( \frac{{V}_{A C}}{{I}_{A C}} ) cos ( {V}_{p} - {I}_{p} ) + j ( \frac{{V}_{A C}}{{I}_{A C}} ) sin ( {V}_{p} - {I}_{p} )$$

where
V_{AC} is the amplitude of the periodic voltage component,
V_{P} is the phase angle of the voltage component,
I_{AC} is the amplitude of the periodic current component,
I_{P} is the phase angle of the current component,
j=√−1, and
Y=1/Z

In the case where nerve tissue is modeled as a bulk parallel RC circuit, R may be obtained for a controlled voltage sinusoidal waveform as follows:

$$R = - {R}_{M} ( \frac{{V}_{M - ma x} + {V}_{M - m i n} + {V}_{A - peak}}{{V}_{M - m ax} + {V}_{M - m i n}} )$$

where
R_{M} is the resistance across the sense resistor,
V_{M-max} is the minimum measured voltage across R_{M},
V_{M-max} is the maximum measured voltage across R_{M}, and
V_{A-peak} is the maximum applied voltage.

As an optional alternative to the methods described above, the best fit parameters may be inferred through analog methods, such as using analog circuit elements. Using analog derived parameters, the electrical characteristics, such as resistance and capacitance, may also be determined.

A display in the various embodiment systems may be any form of electronic display known in the art or that may be developed in the future. Examples of suitable displays that may be used in the various embodiment systems include: a computer screen; a cathode ray tube (CRT); liquid crystal display (LCD); plasma display; arrays of light emitters, e.g., LED; and combinations or variations of these example displays.

The various embodiments may be integrated with image guided procedural equipment that assists clinicians and surgeons by guiding diagnostic, therapeutic and/or surgical instruments to precise locations on a subject or providing clinicians and surgeons with information to enable high precision diagnostic, therapeutic and/or surgical procedures. As used herein, “image-guided equipment” refers to any equipment which positions an instrument or guides an operator to position an instrument based upon patient position information such as contained in an image, such as a CT scan, X-ray, MRI image, ultrasound scan or tissue discrimination scan. Such equipment may be robotic, semi-robotic, tele-robotic in nature, but may also include simple positioning aids such as images projected onto a subject to represent tissues beneath the skin. Since the various embodiment systems may be capable of locating, discriminating and imaging tissues, in particular nerves, this tissue location data may be input into image guided procedural equipment to enable the system to locate, track or avoid sensitive tissues, such as avoiding damaging nerves during invasive procedures or to perform therapeutic or surgical procedures on nerves themselves. For example, clinicians may use image guided equipment employing tissue discrimination data provided by various embodiments to position other imaging technology (e.g., X-ray or ultrasound) on or near certain tissues (e.g., nerves). As another example, anesthesiologists may use image guided equipment employing tissue discrimination data to precisely apply anesthesia to particular nerves without damaging the nerves. As another example, acupuncture needles may be precisely implanted into nerve branches with the aid of patient-relative position information or by means of image-guided equipment. As a further example, surgeons may use image guided equipment employing tissue discrimination data to avoid injuring nerves during surgical procedures.

In the various embodiments, the wave form, amplitude, and duration of an applied signal may all be controlled by the controller. The controller may also send control signals to the multiplexer switching device to provide the generated waveform to a selected waveform electrode 1 for a predefined period of time (a sampling period). Thus, the duration of the applied waveform may be controlled by the microprocessor via the multiplexer switching device or via the waveform generator. In an embodiment, the controller may direct the waveform generator to produce waveforms of a specified amplitude, frequency, and/or shape, e.g., generating a pulsed train or square waveform, a sinusoidal waveform, a sawtooth waveform, etc. Alternatively, the controller may instruct the waveform generator, for example in conjunction with the multiplexer switching device, to apply a plurality of different waveforms, each waveform being applied within a sampling time, to an individual waveform electrode prior to switching to another waveform electrode. Complex waveforms, comprising two or more waveforms of different shape and/or frequency, may also be applied in various embodiments.

The multiplexer switching device may be an electronically controlled switch, a multiplexer, a gate array, or any suitable device that may be controlled by the controller to provide current or voltage from the waveform generator to selected, individual electrodes within the waveform electrode array assembly. In an embodiment, the switching device may be controlled by the controller to apply the generated waveform to a single waveform electrode, to a selected set of electrodes or to all of the waveform electrodes in the array assembly simultaneously. The waveform generator may also be controlled by the controller in association with the switching device to apply the same current to a plurality of waveform electrodes or all of the waveform electrodes independently of each other simultaneously, even when the waveform electrodes experience or exhibit different impedances. The waveform generator and the switching device may also be controlled by the controller to apply a single current or voltage to all of the waveform electrodes or a plurality of waveform electrodes of the waveform electrode array assembly so that the single current or voltage is dispersed among the selected waveform electrodes. Using software executed by the controller to control the waveform, the applied current or voltage may be varied at an individual sample electrode within the array of electrodes, either during one sampling window or after sampling the other electrodes in the array or in a sequential manner.

The controller in the various embodiment systems may be programmed with software that directs the controller to receive commands from an operator to define the parameters of the waveform, e.g., the shape of the waveform, the positive and negative peak amplitudes, the frequency and the duty cycle. The controller may also contain a memory having stored thereon a plurality of predefined waveforms and may select waveforms to be generated by the waveform generator from the predefined set of waveforms. The waveforms may vary in a number of parameters, including for example bias, positive peak amplitude, minimal amplitude, negative peak amplitude, frequency, shape, and/or duty cycle. Controller may alternatively be configured to receive commands from another controller (e.g., a personal computer) electronically connected to the controller, e.g., by a digital data link as known in the art (e.g., Fire Wire, USB, serial or parallel interface, etc.), or by means of a wireless data link transceiver providing a wireless data link as known in the art (e.g., infrared data (IrDA) serial link, IEEE 802.11g, Bluetooth, or similar wireless data link technology as exists or may be developed in the future). In a further embodiment, the electrode array assembly and controller/signal generator may be configured as a wireless component or module configured so that it may be worn by a patient or placed on a patient at a distance from the host computer. In certain hospital environments where electromagnetic radiation may need to be minimized, a standard infrared data link (IrDA) may be preferred. Using a wireless data link between the electrode array assembly and the controller minimizes the impact on other equipment and attending clinicians.

Sampling of signals in the electrode may be continuous, intermittent or periodic. If continuous, it may be detected as a digital signal e.g., via an analog-to-digital (A/D) converter that converts the received analog signal (e.g., voltage or current) into a digital value by integrating the signal over brief sampling windows as is well known in the art.

In the various embodiments, the electrode array assembly may include electrodes configured as wells that may contain the coupling interface material for providing an electrical connection to a subject's skin. When the constituent parts are assembled, the assembly may comprise an array of wells where each well is capped with an electrode, e.g., a gold or silver disk and surrounded by a wall of insulating material formed by aligning an insulating layer with an array of through holes with an array of electrodes so that each cap electrode fits into a single well. Each cap electrode may be electrically connected to a conductor, e.g., by means of a conducting metal paste, with the conductor connected to an electrical coupling that may be coupled to a controller, e.g., a ribbon cable. A return electrode may also be configured to be electrically connected to a subject's skin and located a distance (e.g., about 20 cm.) away from the electrode array assembly device. The coupling interface material for electrodes may be an electrolyte or electrolytic gel, e.g., a hydrophilic, silver-silver chloride gel. In any system where metallic conduction (i.e., wires, flat plates) transitions to ionic conduction in an electrolyte medium (e.g., within tissues), differences in the entities carrying charge for the two media may be considered. In metallic conduction, charge is carried by electrons moving between adjacent electron clouds surrounding the atomic nuclei. In ionic conduction, charge is carried in solution on ions which move toward oppositely charged electrodes. Contact adequacy at the boundary between the metallic phase and the electrolyte phase (e.g., at the skin) determines the efficiency of the transition. This contact ensures the effective exchange of the charge carried by ionic moieties with the metallic surface. In a medical electrode system, the interface medium between the metal (or metal: metal salt) electrode and the skin provides this contact.

To maximize the contact efficiency, the coupling interface material may wet the surfaces of both the electrode and the skin to help reduce the normally high impedance presented by the stratum corneum, and thereby improve electrical conduction through the skin. The coupling interface material may also display a low energy contact that allows the material to spread effectively over the surface, filling any interstices that are present. Thus, the coupling interface material may perform the function of facilitating the conversion of electrical signals from electron-conduction in the electrode to ionic-conduction within tissues. If there is an aqueous medium between the electrode and the skin surface, the energy transfer conversion may occur in this medium. If dry metal electrodes are applied to the skin surface, the transition may occur in the stratum corneum layer of the skin.

Instructions for performing the steps of the methods may be stored in volatile or nonvolatile memory (e.g., PROM or EPROM memory) or on a computer readable medium connected to the controller. A computer readable medium may be any tangible structure, e.g., a magnetic disk, an optical disk, or a magnetic tape; or intangible structure, e.g., a modulated carrier wave containing packetized data, which may be a wireline, optical cable, or a wireless transmission; which is capable of being accessed by a microprocessor or computer. Thus, as used herein, the term “configured to” includes programmed to accomplish or function in the recited manner, as well as physically connected, assembled, wired or otherwise made to accomplish the function.

The controller may be any electronic processing device capable of processing software instructions, receiving data inputs and providing data and command outputs. Examples of suitable processors for use in a system according to the various embodiments include a microprocessor, microcomputer, and microcontroller, as well as external processors/computers, including a personal computer, laptop computer; work station; handheld computer, e.g., a personal data assistant; and combinations or variations of these example processors. A controller or microprocessor may include or be coupled to electronic memory suitable for storing software instructions and data, including volatile and nonvolatile memory as are well known in the art. Data stored in the memory may include the data recorded during operation of the system, and processed data representing tissue discrimination information. The memory may also store data that are useful for operating the system and conducting analysis on measurement data. Data that are useful to an operator for operating the system may include operating instructions, user manuals, trouble-shooting guidance, medical diagnostic guidance, and image interpretation guidance. Such operator-useful information may be stored in the form of a database to provide ready access to a user operating the system. While the operator-useful information may be stored in memory on or near the device (e.g., a hard drive or compact disc reader), it may also be connected to the controller via a network, e.g., the Internet, by suitable communications electronics as more fully described herein.

The test subject may be any tissue, including an external body part such as an arm, or an internal organ of a being. Test subjects (e.g., a human or animal) typically contain at least one electrically responsive membrane system comprising a lipid bi-layer containing embedded protein molecules, some of which are ion channels.

While the aforementioned embodiments employ a digital processor to receive and process sensed electrical parameters to determine the desired electrical characteristic, such as impedance, the various embodiments contemplate the use of analog circuit components to accomplish the same functions. For example, while the signal processing algorithms described herein employ digital sampling and curve fitting algorithms, the same functions may be accomplished by a synchronous demodulator such as employing a phased locked loop circuit element. Thus, the various embodiments are not intended to be limited to the digital components and system described in the example embodiments described herein.

The foregoing description of various embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principles of the invention and its practical application to enable one skilled in the art to utilize the invention in various embodiments and with various modifications as are suited to the particular use contemplated.

What is claimed is:

1. A method for discriminating depth of a nerve beneath a skin surface of a subject, comprising:
placing a waveform electrode array on the skin of the subject, wherein the waveform electrode array comprises at least one waveform electrode that has an area of approximately 10 mm² or less;
placing a return electrode on the skin of the subject at a spacing distance from the at least one waveform electrode such that impedance is minimized, wherein the spacing distance is greater than a distance at which impedance is maximized;
applying at least one electrical signal serially to each of the at least one waveform electrode and the return electrode, wherein an electrical circuit including tissue of the subject as a component is completed;
calculating impedance values of the tissue associated with the applied electrical signal for each of the at least one waveform electrodes;
identifying a waveform electrode with a lowest calculated impedance value;
discriminating a projected (x, y) position of the nerve beneath the waveform electrode array using the identified waveform electrode having a lowest calculated impedance values;
determining a mathematical relationship of impedance (Z) to length (l) for an electrical path to the nerve using known equivalent circuit models;
generating a table correlating impedance with nerve depth, wherein the length of the electrical path to the nerve comprises a sum of a first non-hypotenuse leg and a second non-hypotenuse leg of a right triangle, wherein the first non-hypotenuse leg is equal to a distance from the waveform electrode position on the skin and the projected (x, y) position of the nerve, and wherein the second non-hypotenuse leg is equal to the nerve depth; and
using the table to determine a nerve depth value for the calculated impedance value of a waveform electrode.

2. The method of claim 1, wherein the determined mathematical relationship of impedance (Z) to length (l) for the electrical path to the nerve is based on:
Z_{RLC}∝((1+2l⁴)/2l²)^{-0.5}.

3. The method of claim 1, wherein:
the projected (x, y) position of the nerve comprises one or more coordinates of a path of the nerve in a two-dimensional plane of the waveform electrode array;
determining the projected (x, y) position of the nerve comprises calculating a slope of the nerve path;
the projected (x, y) position of the nerve is determined using impedance values of row waveform electrodes if the slope of the nerve path has an absolute value of greater than or equal to one; and
the projected (x, y) position is determined using impedance values of column waveform electrodes if the slope of the nerve path has an absolute value of less than one.

4. The method of claim 3, further comprising:
determining a total lowest impedance value from the waveform electrode array (Zₘᵢₙ); and
if it is determined that the absolute value of the slope is greater than or equal to one:
identifying a waveform electrode E₁ having an impedance value Z₁ that is a lowest impedance value in comparison to the waveform electrodes in a row with waveform electrode E₁, wherein a difference in impedance between the projected (x, y) position of the nerve and waveform electrode E₁ is ΔZ₁=Z₁−Zₘᵢₙ, and wherein x₀ represents a x-axis coordinate of the projected (x, y) position of the nerve, and the waveform electrode E₁ has a x-axis position of 0;
determining impedance values two waveform electrodes immediately adjacent in the row to, with one on either side of, the waveform electrode E₁;
selecting, from the two waveform electrodes immediately adjacent in the row to the waveform electrode E₁, a waveform electrode E₂ having a lower impedance value Z₂, wherein a difference in impedance between the projected (x, y) position of the nerve and the waveform electrode E₂ is ΔZ₂=Z₂−Zₘᵢₙ, wherein E₂ has a x-axis position of x₂; and
calculating a x-axis coordinate for the projected (x, y) position of the nerve by solving for the represented x₀, wherein:
(x₀−0)/ΔZ₁=(x₂−x₀)/ΔZ₂;
x₀/ΔZ₁=(x₂=x₀)/ΔZ₂;
x₀(ΔZ₂/ΔZ₁)=x₂−x₀;
x₀(1+ΔZ₂/ΔZ₁)=x₂; and
x₀=x₂/(1+ΔZ₂/ΔZ₁).

5. The method of claim 3, wherein calculating the slope of the nerve path comprises:
determining waveform electrodes A₁ and A₂ that are waveform electrodes having the lowest impedance values in comparison to all of the waveform electrodes, wherein coordinates of A₁ in the waveform electrode array are (x_{A1}, y_{A1}), and wherein coordinates of A₂ in the electrode array are (x_{A2}, y_{A2});
calculating a value M, wherein
$$M = \frac{( {y}_{A 2} - {y}_{A 1} )}{( {x}_{A 2} - {x}_{A 1} )} ;$$
determining an electrode A₂ that is a waveform transverse slope for each waveform electrode, wherein the transverse slope is determined by calculating a difference between impedance values at adjacent row waveform electrodes on either side, and dividing the difference by a linear distance D_{T} between the adjacent row waveform electrodes;

6. The method of claim 1, wherein the at least one electrical signal employs a single frequency between approximately 100 Hz and approximately 10,000 Hz.

7. The method of claim 1, further comprising:
measuring a change in a characteristic of the applied electrical signal resulting from transmission through tissue between the waveform and return electrodes; and
processing the measured change in the characteristic to discriminate features of the nerve located beneath the waveform electrode.

8. The method of claim 7, wherein the characteristic is voltage.

9. The method of claim 7, wherein the characteristic is current.

10. The method of claim 1, wherein one of the at least one electrical signal is periodic.

11. The method of claim 1, wherein one of the at least one electrical signal is aperiodic.

12. The method of claim 1, further comprising:
determining whether the calculated impedance values are affected by electrical resonance of the applied electrical signal;
generating a new electrical signal at a new frequency; if it is determined that the calculated impedance values are affected by electrical resonance;
applying the new electrical signal to each of the at least one waveform electrode and the return electrode; and
re-calculating the impedance values of the tissue associated with the applied new electrical signal for each of the at least one waveform electrodes.

13. The method of claim 12, wherein determining whether the calculated impedance values are affected by electrical resonance of the applied electrical signal comprises:
applying a square waveform, controlled current pulse to the tissue;
creating a voltage decay curve for the controlled current, square waveform applied to the tissue;
extracting constituent time constants from the voltage decay curve;
applying the electrical signal to the tissue at a selected frequency, wherein the applied electrical signal is a periodic waveform;
determining, for the at least one waveform electrode that demonstrates a longest constituent first time constant, a first impedance value at the selected frequency; and
comparing the longest constituent first time constant with the first impedance value.

14. The method of claim 13, wherein extracting constituent time constants from the voltage decay curve is performed by logarithmic stripping.

15. The method of claim 13, further comprising using logarithmic stripping to identify the at least one waveform electrode that demonstrates the longest first constituent time constant.

16. The method of claim 12, further comprising comparing at least two characteristics of the applied electrical signal to discriminate a location of anisotropic features associated with the nerve beneath the waveform electrode array.

17. The method of claim 1, further comprising using the projected (x, y) position of the nerve and the determined nerve depth value to generate an image of a discriminated location of the nerve in (x, y, z) space beneath the waveform electrode array

18. The method of claim 17, further comprising displaying the generated image on a display device.

19. The method of claim 17, wherein the generated image of the discriminated location of the nerve tissue is used to generate a data set representing the discriminated location of the nerve tissue, and wherein the method further comprises storing the data set in a database.

20. The method of claim 1, wherein placing a waveform electrode array on the skin of the subject comprises placing a plurality of waveform electrode arrays on a plurality of locations on the skin of the subject, the method further comprising:
recording the location of each of the plurality of waveform electrode arrays; and
using discriminated locations of the nerve tissue associated with each of the plurality of waveform electrode arrays to generate a plurality of discriminated locations of nerve tissue beneath the skin of the subject.

21. The method of claim 1, wherein the spacing distance between the at least one waveform electrode and the return electrode is approximately 20 cm.

22. A tissue discrimination system, comprising:
a waveform generator configured to generate a plurality of different waveforms;
a waveform electrode array coupled to the waveform generator, wherein the waveform electrode array comprises at least one waveform electrode that is at least 10 mm², and wherein the waveform electrode array is configured to apply a waveform to a tissue;
at least one return electrode configured to receive the applied waveform from the tissue and to provide the applied waveform to the controller, wherein the return electrode is spaced at an inter-electrode distance from the waveform electrode array, wherein the inter-electrode distance minimizes impedance and is greater than a distance of maximum impedance; and
a controller coupled to the waveform generator and the at least one return electrode, wherein the controller is configured to perform operations comprising:
causing a waveform to be applied serially to the waveform electrode array and the return electrode,
calculating impedance of the tissue associated with the applied waveform for each of the at least one waveform electrodes;
identifying a waveform electrode with a lowest calculated impedance value;
discriminating a projected (x, y) position of the nerve beneath the waveform electrode array using the identified waveform electrode having a lowest calculated impedance values;
determining a mathematical relationship of impedance (Z) to length (l) for an electrical path to the nerve using known equivalent circuit models;
generating a table correlating impedance with nerve depth, wherein the length of the electrical path to the nerve comprises a sum of a first non-hypotenuse leg and a second non-hypotenuse leg of a right triangle, wherein the first non-hypotenuse leg is equal to a distance from the waveform electrode position on the skin and the projected (x, y) position of the nerve, and wherein the second non-hypotenuse leg is equal to the nerve depth; and
using the table to determine a nerve depth value for the calculated impedance value of a waveform electrode.

23. The tissue discrimination system of claim 22, further comprising:
a sensor circuit coupled to the controller, waveform electrode array and return electrode, wherein the controller is configured to perform operations further comprising:
receiving a signal from the sensor circuit; and
calculating electrical parameters using the received signal.
